(19) United States
(12) Patent Application Publication
(10) Pub. No.: US 2010/0015078 A1
(43) Pub. Date: Jan. 21, 2010

(54) CONDITIONING COMPOSITION COMPRISING DUAL CATIONIC SURFACTANT SYSTEM, AMINOSILICONE AND SILICONE RESIN

(76) Inventor: Yujun Li, Kobe (JP)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/175,697

(22) Filed: Jul. 18, 2008

(51) Int. Cl. A61K 8/84; A61Q 5/12

(52) U.S. Cl. 424/70.12

(57) ABSTRACT

Disclosed is a conditioning composition comprising: (a) from about 0.1% to about 10% of a surfactant system comprising: di- and mono-alkyl quaternized ammonium salt cationic surfactants; (b) from about 1% to about 15% of a high melting point fatty compound; (c) from about 0.1 % to about 20% of an aminosilicone; (d) from about 0.0001% to about 10% of a silicone resin; and (e) an aqueous carrier. The composition of the present invention can provide improved wet and dry conditioning benefits while providing chronic/long lasting color protection benefits.

### FIELD OF THE INVENTION

The present invention relates to a conditioning composition comprising: (a) a surfactant system comprising: di- and mono-alkyl quaternized ammonium salt cationic surfactants; (b) a high melting point fatty compound; (c) an aminosilicone; (d) a silicone resin; and (e) an aqueous carrier. The composition of the present invention can provide improved wet and dry conditioning benefits while providing durable conditioning and chronic/long lasting color protection benefit.

### BACKGROUND OF THE INVENTION

A variety of approaches have been developed to condition the hair. A common method of providing conditioning benefit is through the use of conditioning agents such as cationic surfactants and polymers, high melting point fatty compounds, low melting point oils, silicone compounds, and mixtures thereof. Most of these conditioning agents are known to provide various conditioning benefits. For example, some cationic surfactants, when used together with some high melting point fatty compounds, are believed to provide a gel matrix which is suitable for providing a variety of conditioning benefits such as slippery feel during the application to wet hair and softness and moisturized feel on dry hair.

There is a need for hair conditioning compositions which provide durable conditioning and chronic/long lasting color protection benefit while providing improved wet and dry conditioning benefits. Such durable conditioning and chronic/long lasting color protection benefits are, for example, at least one of the followings: maintaining good sensory feel long period after coloring hair, preventing color fading of colored hair or minimizing/slowing down color lost until next coloring hair.

WO publication No. WO 2005/107682 discloses the conditioning of hair after it has been oxidized in a coloring, bleaching or perming treatment, which comprises conditioning treatment with a conditioning compositions comprising a functionalized silicone such as aminosilicones. WO publication No. WO 2005/107682 also discloses, in Example 3, a hair conditioner comprising: stearamidopropyl dimethylamine; dicetyl dimethyl ammonium chloride; cetyl alcohol; stearyl alcohol; amino-silicone having a tradename TSF4707 and MQ resin having a tradename SR1000.

However, it has been found that; it is still not easy to obtain hair conditioning compositions which provide improved wet and dry conditioning benefits while providing durable conditioning and chronic/long lasting color protection benefit.

Based on the foregoing, there remains a need for conditioning compositions which provide improved wet and dry conditioning benefits while providing durable conditioning and chronic/long lasting color protection benefit.

None of the existing art provides all of the advantages and benefits of the present invention.

### SUMMARY OF THE INVENTION

The present invention is directed to a conditioning composition comprising by weight:
(a) from about 0. 1% to about 10% of a surfactant system comprising:

a dialkyl quaternized ammonium salt cationic surfactant; and

a monoalkyl quaternized ammonium salt cationic surfactant;
(b) from about 1% to about 15% of a high melting point fatty compound;
(c) from about 0.1 % to about 20% of an aminosilicone;
(d) from about 0.0001% to about 10% of a silicone resin; and
(e) an aqueous carrier.

The conditioning compositions of the present invention can provide improved wet and dry conditioning benefits while providing durable conditioning and chronic/long lasting color protection benefits.

These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

### Composition

It is believed that; the combination of (i) the cationic surfactant system comprising di- and mono-alkyl quaternized ammonium salt cationic surfactants and (ii) the silicone system comprising aminosilicones and silicone resins, together with other required elements of the present invention, provides improved wet and dry conditioning benefits while providing durable conditioning and chronic/long lasting color protection benefit.

Preferably, when containing cationic surfactants and/or gel matrix formed by cationic surfactants and high melting point fatty compounds, the composition of the present invention is substantially free of anionic surfactants and anionic polymers, in view of avoiding undesirable interaction with cationic surfactants and/or in view of stability of the gel matrix. In the present invention, “substantially free of anionic surfactants and anionic polymers” means that the composition contains 1% or less, preferably 0.5% or less, more preferably totally 0% of total of anionic surfactants and anionic polymers.

### Cationic Surfactant System

The composition of the present invention comprises a cationic surfactant system comprising a dialkyl quaternized ammonium salt cationic surfactant and a monoalkyl quaternized ammonium salt cationic surfactant. The cationic surfactant system is included in the composition at a level by weight of from about 0.1% to about 10%, preferably from about 0.3% to about 5%, more preferably from about 0.5% to about 3%.

It is believed that; the combination of dialkyl and monoalkyl quatemized ammonium salt cationic surfactants can provide improved deposition of the silicones and fatty alcohols of the present invention, compared to other cationic surfactants. Such other cationic surfactants are, for example, other combinations of cationic surfactants or use of a single cationic surfactant, such other combinations including that of a dialkyl quaternized ammonium salt cationic surfactant and an amidoamine cationic surfactant such as stearamidopropyl dimethylamine, behenamidopropyl dimethylamine, and quaternized with glutamic acid.

In view of improved deposition of such silicones, it is preferred to contain cationic surfactant system such that the weight ratio of the dialkyl quaternized ammonium salt cationic surfactant to the monoalkyl quaternized ammonium salt cationic surfactant is within the range of from about 1:1 to about 1:20, more preferably from about 1:1 to about 1:15, still more preferably from about 1:1 to about 1:10,

### (i) Dialkyl Quaternized Ammonium Salt Cationic Surfactant

Dialkyl quaternized ammonium salt cationic surfactants useful herein are those having the formula:

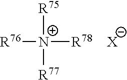

wherein two of R⁷⁵, R⁷⁶, R⁷⁷ and R⁷⁸ are selected from an alkyl group of from 12 to 30 carbon atoms or an aromatic, alkoxy, polyoxyalkylene, alkylamido, hydroxyalkyl, aryl or alkylaryl group having up to about 30 carbon atoms with or without an ester group; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated or branched. Preferably, two of R⁷⁵, R⁷⁶, R⁷⁷ and R⁷⁸ are selected from an alkyl group of from 12 to 30 carbon atoms, more preferably from 16 to 22 carbon atoms, still more preferably from 16 to 20 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Nonlimiting examples of preferred dialkyl quaternized ammonium salt cationic surfactants include: dicocodimonium chloride; dicocodimonium bromide; dimyristyldimonium chloride; dimyristyldimonium bromide; dicetyldimonium chloride; dicetyldimonium bromide; dicetylmethylbenzylmonium chloride; distearyldimonium chloride; distearyldimonium bromide; dimetyldi(hydrogenated tallow)monium chloride; hydroxypropylbisstearylmonium chloride; distearylmethylbenzylmonium chloride; dibehenyl/diarachidyldimonium chloride; dibehenyl/diarachidyldimonium bromide; dibehenyldimonium chloride; dibehenyldimonium bromide; dibehenyldimonium methosulfate; dibehenylmethylbenzylmonium chloride; dihydrogenated tallow benzylmonium chloride; dihydrogenated tallowethyl hydroxyethylmonium methosulfate; dihydrogenated tallow hydroxyethylmonium methosulfate; di-C12-15 alkyldimonium chloride; di-C12-18 alkyldimonium chloride; di-C14-18 alkyldimonium chloride; dicocoylethyl hydroxyethylmonium methosulfate; disoyoylethyl hydroxyethylmonium methosulfate; dipalmitoylethyldimonium chloride; dihydrogenated palmoylethyl hydroxyethylmonium methosulfate; dihydrogenated tallowamidoethyl hydroxyethylmonium chloride; dihydrogenated tallowamidoethyl hydroxyethylmonium methosulfate; dihydrogenated tallowoylethyl hydroxyethylmonium methosulfate; distearoylethyl hydroxyethylmonium methosulfate; Quaternium-82.

Another dialkyl quarternized ammonium salt cationic surfactant useful herein asymmetric dialkyl quarternized ammonium salt cationic surfactants. It is believed that the asymmetric dialkyl quarternized ammonium salt cationic surfactant can provide easy-to rinse feel, compared to symmetric dialkyl quaternized ammonium salt cationic surfactants. The asymmetric dialkyl quaternized ammonium salt cationic surfactants useful herein are those having the formula:

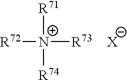

wherein R⁷¹ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; R⁷² is selected from an alkyl group of from 5 to 12 carbon atoms or an aromatic, alkoxy, polyoxyalkylene, alkylamido, hydroxyalkyl, aryl or alkylaryl group having up to about 12 carbon atoms; R⁷³ and R⁷⁴ are independently selected from an alkyl group of from 1 to about 4 carbon atoms or an aromatic, alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether linkages, ester linkages, and other groups such as amino groups. The longer chain alkyl groups, e.g., those of about 12 carbons, or higher, can be saturated or unsaturated and/or straight or branched. Preferably, R⁷¹ is selected from a non-functionalized alkyl group of from 12 to 30 carbon atoms, preferably from 16 to 22 carbon atoms; R⁷² is selected from a non-functionalized alkyl group of from 5 to 12 carbon atoms, more preferably from 6 to 10 carbon atoms, still more preferably 8 carbon atoms; R⁷³ and R⁷⁴ are independently selected from CH₃, C₂H₅, C₂H₄OH, and mixtures thereof; and X is selected from the group consisting of Cl, Br, CH₃OSO₃, C₂H₅OSO₃, and mixtures thereof. More preferably, R⁷¹ is a straight, saturated non-functionalized alkyl group, and R⁷² is a branched saturated non-functionalized alkyl group. Still more preferably, the branched group of R⁷² is a straight, saturated alkyl group of from 1 to 4 carbon atoms, even more preferably 2 carbon atoms.

Nonlimiting examples of preferred asymmetric dialkyl quaternized ammonium salt cationic surfactants include: stearylethylhexylmonium chloride, stearylethylhexylmonium bromide; stearyl ethylhexyl dimonium methosulfate; cetearyl ethylhexyldimonium Methosulfate available, for example, with tradename Varisoft ASQ from Evonik.

### (ii) Monoalkyl Quaternized Ammonium Salt Cationic Surfactant

Mono-alkyl quaternized ammonium salt cationic surfactants useful herein are those having the formula:

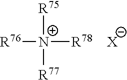

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 22 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof. It is also believed that the use of alkylsulfate such as methosulfate and ethosulfate as a salt-forming anion may be able to provide better conditioning benefits especially wet conditioning benefits, compared to other salt-forming anions.

Nonlimiting examples of such monoalkyl quaternized ammonium salt cationic surfactants include: behenyl trimethyl ammonium salt such as behenyl trimethyl ammonium methosulfate and behenyl trimethyl ammonium chloride; stearyl trimethyl ammonium salt; cetyl trimethyl ammonium salt; and hydrogenated tallow alkyl trimethyl ammonium salt.

### High Melting Point Fatty Compound

The composition of the present invention comprises a high melting point fatty compound. The high melting point fatty compound is included in the composition at a level of from about 1% to about 15%, preferably from about 2% to about 10%, more preferably from about 3% to about 8% by weight of the composition, in view of providing improved conditioning benefits such as slippery feel during the application to wet hair, softness and moisturized feel on dry hair.

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about 22 carbon atoms. These fatty alcohols are saturated and can be straight or branched chain alcohols. Preferred fatty alcohols include, for example, cetyl alcohol, stearyl alcohol, behenyl alcohol, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

### Aqueous Carrier

The conditioning composition of the present invention comprises an aqueous carrier. The level and species of the carrier are selected according to the compatibility with other components, and other desired characteristic of the product. Generally, the compositions of the present invention comprise from about 20% to about 99%, preferably from about 30% to about 95%, and more preferably from about 80% to about 95% water.

The carrier useful in the present invention includes water and water solutions of lower alkyl alcohols and polyhydric alcohols. The lower alkyl alcohols useful herein are monohydric alcohols having 1 to 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product.

### Gel Matrix

Preferably, the above cationic surfactants, together with high melting point fatty compounds and an aqueous carrier, form a gel matrix in the composition of the present invention.

The gel matrix is suitable for providing various conditioning benefits such as slippery feel during the application to wet hair and softness and moisturized feel on dry hair. In view of providing the above gel matrix, the cationic surfactant and the high melting point fatty compound are contained at a level such that the weight ratio of the cationic surfactant to the high melting point fatty compound is in the range of, preferably from about 1:1 to about 1:10, more preferably from about 1:1 to about 1:6.

For forming gel matrix, it is preferred to prepare the composition by the following method:

Water is typically heated to at least about 70° C., preferably between about 80° C. and about 90° C. The cationic surfactant and the high melting point fatty compound are combined with the water to form a mixture. The temperature of the mixture is preferably maintained at a temperature higher than both the melting temperature of the cationic surfactant and the melting temperature of the high melting point fatty compound, and the entire mixture is homogenized. After mixing until no solids are observed, the mixture is gradually cooled (e.g., at a rate of from about 1° C./minute to about 5° C./minute) to a temperature below 60° C., preferably less than about 50° C. During this gradual cooling process, a significant viscosity increase is observed at between about 50° C. and about 60° C. This indicates the formation of gel matrix. Additional components are then combined with the gel matrix, and cooled to room temperature.

Preferably, the present invention comprises, by weight of the composition, from about 60% to about 99%, preferably from about 70% to about 95%, and more preferably from about 80% to about 95% of a gel matrix, to which optional ingredients such as silicones can be added. The composition containing the above amount of gel matrix is typically characterized by rheology at 950s-1 of from about 40 Pa to about 600 Pa, preferably from about 50 Pa to about 500 Pa, and more preferably from about 70 Pa to about 400 Pa, as measured at 26.7° C., by means of TA AR1000 rheometer at shear rate from 0.1 s⁻¹ to 1100 s⁻¹ with the duration of 1 minutes. Although the composition of the present invention can contain a thickening polymer, the composition of the present invention can have the above rheology with the presence of any thickening polymer. Although the composition of the present invention may contain a thickening polymer, the composition of the present invention can have the above viscosity without the presence of any thickening polymer.

### Aminosilicone

The compositions of the present invention comprise an aminosilicone. The aminosilicone is included in the composition at levels by weight of the composition of from about 0.1% to about 20%, preferably from about 0.25% to about 15%, more preferably from about 0.5% to about 10%, still more preferably from about 1% to about 7%.

The aminosilicone useful herein include, but are not limited to silicones of the following structure:

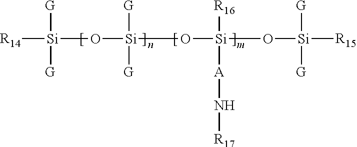

wherein:
a sum (n+m) ranges from about 2 to about 2000, preferably from about 150 to about 2000, more preferably from about 250 to about 1200, still more preferably from about 300 to about 800; n is a number ranging from about 1 to about 1999, and m is a number ranging from about 1 to about 1999; and n and m are chosen such that a ratio of m:n is from about 1:1000 to about 1:10, preferably from about 1:1000 to about 1:25, more preferably from about 1:800 to about 1:50, still more preferably from about 1:500 to about 1:50, even more preferably from about 1:400 to about 1:100;
R₁₄, R₁₅, R₁₆, which may be identical or different, are chosen from a hydroxyl radical, C1-C4 alkoxy radicals and methyl, preferably R₁₄ and R₁₅ are hydroxyl radical and/or C1-C4 alkoxy radicals and R₁₆ is methyl;
A is chosen from linear and branched C3-C8 alkenyl radicals;
R₁₇ is chosen from H, phenyl, linear or branched C1-C4 alkyl radical, benzyl or preferably linear or branched (C2-C8)NH₂; and
G is chosen from H, phenyl, hydroxyl, C1-C8 alkyl, preferably methyl.
These aminosilicones may be of the random or block type.

Suitable aminosilicones of the present invention include, but are not limited to, organomodified silicones with amine functionality available commercially under the trade names such as ADM1100 and ADM1600 from Wacker Silicones, AP6087, DC2-8211, DC8822, DC8822A, DC8803, DC2-8040, DC2-8813, DC2-8630 and DC8566 from Dow Corning Corporation, KF-862, KF-861, KF-862S, KF-8005, KF-8004, KF-867S, KF-873, and X-52-2328 from Shin-Etsu Corporation, and TSF 4702, TSF 4703, TSF 4704, TSF 4705, TSF 4707, TSF 4708, TSF 4709, F42-B3115, SF 1708, SF 1923, SF 1921, SF 1925, OF TP AC3309, OF 7747, OF-NH TP A13631, OF-NH TP A13683 from GE Bayer Silicones.

Highly preferred aminosilicones of the present invention are organomodified silicones with amine functionality with viscosities of greater than about 4,000 mPa·s in view of conditioning efficiency and up to about 100,000 mPa·s in view of friendly incorporation processing and spreadability, which include, but are not limited to, commercially available fluids under the trade names ADM1100 from Wacker Silicones, AP6087, DC8803 from Dow Coming Corporation, and TSF 4707 from GE Bayer Silicones.

### Silicone Resin

The compositions of the present invention comprise a silicone resin. Without wishing to be bound by theory, silicone resins are believed to create a 3-dimensional network within the aminosilicone fluid giving rise to vicoelasticity thereby improving the adhesive properties of the fluid and hence the durability on a fibrous substrate. Preferably, the silicone resin is insoluble in water. In the case that the fiber treatment composition is an emulsion, the mixture of the aminosilicone and the silicone resin may be dispersed therewithin in the form of emulsified droplets.

Preferably, the organosiloxane resins according to the invention are solid at about 25° C. Whilst not wishing to be bound to theory, it is believed that solid silicone resin can form an ultrafine dispersion in the aminosilicone which behaves unlike any of the silicone resin per se, the aminosilicone per se, and a blended fluid with the aminosilicone when using fluid silicone resin.

Preferably, the organosiloxane resins according to the invention have a molecular weight range of from about 500 to about 50,000, more preferably from about 750 to about 25,000, still more preferably from about 1,000 to about 10,000 grams/mole. Whilst not wishing to be bound to theory, it is believed that silicone resins of lower or larger molecular weight tend to provide reduced synergy with the aminosilicone.

The silicone resin is included in the composition at levels by weight of the composition of from about 0.0001% to about 10%, preferably from about 0.001% to about 5%, more preferably from about 0.002% to about 3%, still more preferably from about 0.003% to about 1%. It is preferred to contain the silicone resin such that the weight ratio of the silicone resin to the aminosilicone is within the scope of from about 1:500 to about 1:3000, more preferably from about 1:800 to about 1:2000, still more preferably from about 1:800 to about 1:1500. It is believed that a lower level of the silicone resin provides reduced synergetic benefit with the aminosilicone, while a higher level of the silicone resin tend to provide poor sensory feel.

Organosiloxane resins useful herein are combinations of R₃SiO_{1/2} “M” units, R₂SiO “D” units, RsiO_{3/2} “T” units, SiO₂ “Q” units in ratios to each other that satisfy the relationship RnSiO_{(4-n)/2} where n is a value between 1.0 and 1.50 and R is a methyl group. Silanol or alkoxy functionalities may also be present in the resin structure.

More preferably, the organosiloxane resins comprise repeating monofunctional R₃SiO_{1/2} “M” units and the quadrafunctional SiO₂ “Q” units, otherwise known as “MQ” resins. In this case, the ratio of the “M” to “Q” functional units is advantageously from 0.7 and the value of n is 1.2. Organosiloxane resins such as these are commercially available as SRI000 available from GE Bayer Silicones and Wacker 803 from Wacker Silicones.

### Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: low melting point oils having a melting point of less than 25° C. including, for example, unsaturated fatty alcohols such as oleyl alcohol and ester oils such as pentaerythritol ester oils; other oils such as safflower seed oil, coconut oil, cationic conditioning polymers including, for example, cationic celluloses and cationic guar gums; polyethylene glycols; other conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, hydrolyzed sweet almond protein, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; perfumes; and sequestering agents, such as disodium ethylenediamine tetra-acetate; ultraviolet and infrared screening and absorbing agents such as octyl salicylate, octyl methoxycinnamate, benzophenone-3 and benzophenone-4; and antidandruff agents such as zinc pyrithione and salicylic acid.

### Product Forms

The conditioning compositions of the present invention can be in the form of rinse-off products or leave-on products, and can be formulated in a wide variety of product forms, including but not limited to creams, gels, emulsions, mousses and sprays.

The conditioning composition of the present invention is especially suitable for rinse-off hair conditioner. Such compositions are preferably used with a colorant composition by following steps:
(i) applying a colorant composition to the hair for a period of time between 5 and 60 min;
(ii) optionally and preferably rinsing the hair with water;
(iii) then, applying an effective amount of the conditioning composition to the hair; and
(iv) preferably rinsing the hair with water.
   Such conditioning compositions are also used by following steps:
(i) after shampooing hair, applying to the hair an effective amount of the conditioning compositions for conditioning the hair; and
(ii) then rinsing the hair.

### Examples

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

### [Compositions]

| Components | Ex. 1 | Ex. 2 | Ex. 3 | Ex. 4 | Ex. 5 | Ex. 6 | Ex. 7 | Ex. 8 |
|---|---|---|---|---|---|---|---|---|
| Dicetyldimethylammonium chloride | 0.35 | 0.35 | 0.35 | 0.35 | — | — | — | 0.35 |
| Distearyldimethylammonium chloride | — | — | — | — | 0.35 | — | — | — |
| Distearoylethyl hydroxyethylmonium | — | — | — | — | — | — | 0.35 | — |
| methosulfate *1 | | | | | | | | |
| Asymmetric dialkyl quaternized | — | — | — | — | — | 0.9 | — | — |
| ammonium salt cationic surfactant *2 | | | | | | | | |
| Behenyl trimethyl ammonium chloride | — | — | — | — | 2.25 | — | — | — |
| Behenyl trimethyl ammonium | 1.1 | 1.1 | 1.1 | 1.1 | — | 1.4 | 1.8 | 1.8 |
| methosulfate | | | | | | | | |
| Cetyl alcohol | 0.9 | 0.9 | 0.9 | 0.9 | 1.9 | 1.9 | 1.5 | 1.5 |
| Stearyl alcohol | 2.3 | 2.3 | 2.3 | 2.3 | 3.6 | 4.6 | 3.7 | 3.7 |
| Behenyl alcohol | — | — | — | — | 1.0 | — | — | — |
| Aminosilicone-1 *3 | 5.0 | 3.5 | — | — | 7.0 | 5.0 | 5.0 | 3.5 |
| Aminosilicone-2 *4 | — | — | 5.0 | 3.5 | — | — | — | — |
| MQ resin *5 | 0.005 | 0.0035 | 0.006 | 0.004 | 0.007 | 0.005 | 0.005 | 0.004 |
| Isopropanol | 0.3 | 0.3 | 0.3 | 0.3 | 0.3 | 0.3 | 0.3 | 0.3 |
| Preservatives | 0.6 | 0.6 | 0.6 | 0.6 | 0.6 | 0.6 | 0.6 | 0.6 |
| Perfume | 0.4 | 0.4 | 0.4 | 0.4 | 0.4 | 0.4 | 0.4 | 0.4 |
| Panthenol | 0.03 | — | — | — | 0.03 | 0.03 | — | — |
| Panthenyl ethyl ether | 0.03 | — | — | — | 0.03 | 0.03 | — | — |

| | |
|---|---|
| Deionized Water | q.s. to 100% |

Definitions of Components
*1 Distearoylethyl hydroxyethylmonium methosulfate: Dehyquart F-75 from Cognis.
*2 Asymmetric dialkyl quatemized ammonium salt cationic surfactant-1: Cetearyl ethylhexyl
dimonium methosulfate, available with a tradename Varisoft ASQ from Evonik
*3 Aminosilicone-1: having the following structure:

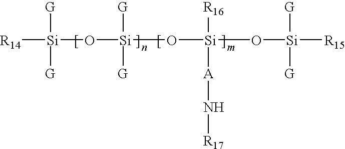

wherein: m and n are numbers with a sum (n + m) ranging from 300 to 800 and are chosen such that
ratio of m:n is from 1:400 to 1:100; R₁₄ and R₁₅ are hydroxyl radical and/or C1-C4 alkoxy radicals
and R₁₆ is methyl; A is chosen from linear and branched C3-C8 alkenyl radicals; R₁₇ is chosen from
linear or branched (C2-C8)NH₂; and G is methyl.
*4 Aminosilicone-2: ADM1100 from Wacker Silicones
*5 MQ resin: SR1000 (Polytrimethyl hydrosilylsilicate) from GE Silicones

### Method of Preparation

The conditioning compositions of “Ex. 1” through “Ex. 8” as shown above can be prepared by any conventional method well known in the art. They are suitably made as follows:

Cationic surfactants and high melting point fatty compounds are added to water with agitation, and heated to about 80° C. The mixture is cooled down to about 50° C. If included, silicone compounds, perfumes, preservatives are added to the mixture with agitation. Then the mixture is cooled down to room temperature.

Examples 1 through 8 are hair conditioning compositions of the present invention which are particularly useful for rinse-off use. The embodiments disclosed and represented by the previous “Ex. 1” through “Ex. 8” have many advantages. For example, they can provide improved wet and dry conditioning benefits while providing durable conditioning and chronic/long lasting color protection benefits.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A conditioning composition comprising by weight:
(a) from about 0.1% to about 10% of a surfactant system comprising:
a dialkyl quaternized ammonium salt cationic surfactant; and
a monoalkyl quaternized ammonium salt cationic surfactant;
(b) from about 1% to about 15% of a high melting point fatty compound having a melting point of 25° C. or higher;
(c) from about 0.1% to about 20% of an aminosilicone;
(d) from about 0.0001% to about 10% of a silicone resin; and
(e) an aqueous carrier.

2. The conditioning composition of claim 1 wherein the weight ratio of the dialkyl quaternized ammonium salt cationic surfactant to the monoalkyl quaternized ammonium salt cationic surfactant is within the range of from about 1:1 to about 1:20.

3. The conditioning composition of claim 1 wherein the monoalkyl quaternized ammonium salt cationic surfactant comprises an alkylsulfate anion selected from methosulfate or ethosulfate.

4. The conditioning composition of claim 1 wherein the silicone resin is solid at about 25° C. and has a molecular weight range of from about 1,000 to about 10,000 grams.

5. The conditioning composition of claim 1 wherein the weight ratio of the silicone resin to the aminosilicone is within the scope of from about 1:500 to about 1:3000.

6. The conditioning composition of any of claim 1 wherein the composition is substantially free of anionic surfactants and anionic polymers.

7. The conditioning composition of claim 1 which is a rinse-off hair conditioning composition.
